(19) United States
(12) Patent Application Publication
(10) Pub. No.: US 2014/0364749 A1
(43) Pub. Date: Dec. 11, 2014

(54) CALIBRATION FOR BLOOD PRESSURE MEASUREMENTS

(71) Applicants: Kushal Varma, Philadelphia, PA (US); Ahmadreza Pourshoghi, Ardmore, PA (US); Nathaniel Magee, Philadelphia, PA (US); Atman Shah, Wexpord, PA (US); Sagar Shah, San Diego, CA (US); Marek Swoboda, Philadelphia, PA (US); Ryszard M. Lec, Philadelphia, PA (US)

(72) Inventors: Kushal Varma, Philadelphia, PA (US); Ahmadreza Pourshoghi, Ardmore, PA (US); Nathaniel Magee, Philadelphia, PA (US); Atman Shah, Wexpord, PA (US); Sagar Shah, San Diego, CA (US); Marek Swoboda, Philadelphia, PA (US); Ryszard M. Lec, Philadelphia, PA (US)

(73) Assignee: Drexel University, Philadelphia, PA (US)

(21) Appl. No.: 13/880,382

(22) PCT Filed: Oct. 21, 2011
(86) PCT No.: PCT/US11/57280

§ 371 Date: Dec. 5, 2013

Related U.S. Application Data
(60) Provisional application No. 61/405,620, filed on Oct. 21, 2010.

(51) Int. Cl. A61B 5/022; A61B 5/00; A61B 5/021

(52) U.S. Cl. CPC A61B 5/02233; A61B 5/02125; A61B 5/7278; A61B 5/681
USPC 600/494; 600/499

(57) ABSTRACT

Blood pressure measurement devices comprising a transducer may have error introduced when the transducer is placed in mechanical communication with a vein or artery. This error may be based on alignment, applanation, calibration, and the contact based stress required to obtain a signal from pressure in a vein or artery. The present invention teaches isolating and removing this error from the blood pressure system which may increase the accuracy of the measurement. Calibration is also provided.

### BACKGROUND

Blood pressure measurement is a staple of the medical profession. Known blood pressure measurement methods such as the auscultatory method and the oscillometric typically require the use of an inflatable cuff, and, as in the case of oscillometric methods, strict calibration of equipment is also required.

The auscultatory method uses a stethoscope and a sphygmomanometer. This method comprises an inflatable cuff placed around the upper arm at roughly the same height as the heart, attached to a mercury or aneroid manometer. The inflatable cuff is fitted and then inflated until the artery is completely occluded. Listening with the stethoscope to the brachial artery at the elbow, the listener slowly releases the pressure in the inflatable cuff. When blood just starts to flow in the artery, the turbulent flow creates a “whooshing” or pounding (first Korotkoff sound). The pressure at which this sound is first heard is the systolic blood pressure. The inflatable cuff pressure is further released until no sound can be heard (fifth Korotkoff sound), at the diastolic arterial pressure.

The auscultatory method, however, typically requires the presence of a medical professional and does not allow for continuous monitoring of the blood pressure of a patient. Further, the inflatable arm cuff and other elements of the system can be bulky and obtrusive.

Another method of measuring blood pressure is the oscillometric method. The equipment used in the oscillometric method is functionally similar to that of the auscultatory method, but the method uses an electronic pressure sensor instead of the stethoscope and an expert's ear. In practice, the pressure sensor is a calibrated electronic device with a numerical readout of blood pressure. In most cases a cuff is inflated and released by an electrically operated pump and valve, which can be fitted on the wrist (elevated to heart height), although the upper arm is preferred. Values of systolic and diastolic pressure are computed, not actually measured, and the computed results are displayed.

In the oscillometric method, a cuff is inflated to a pressure initially in excess of the systolic arterial pressure, and then reduces to below diastolic pressure over a period of about 30 seconds. When blood flow is nil (inflatable cuff pressure exceeding systolic pressure) or unimpeded (inflatable cuff pressure below diastolic pressure), cuff pressure will be essentially constant. It is essential that the inflatable cuff size is correct: undersized cuffs can yield too high a pressure, whereas oversized cuffs yield too low a pressure. When blood flow is present, but restricted, the cuff pressure, which is monitored by the pressure sensor, will vary periodically in synchrony with the cyclic expansion and contraction of the brachial artery, i.e., it will oscillate.

The oscillometric method, like the auscultatory method, requires the use of an inflatable cuff and has the further problem of calibration. Further, the oscillometric method typically takes individual blood pressure measurements and continuous monitoring of blood pressure generally not achievable with this method. The devices that use the oscillometric method also tend to be bulky and inaccurate.

Invasive methods of blood pressure measurements are also known in the art; however, these require either implantation or otherwise piercing the skin. They are typically performed by medical professionals and have discomfort and other potential complications associated with them.

Hydrostatic pressure refers to the force that a liquid exerts against the walls of its container. In terms of blood pressure, it is the amount of pressure that the blood causes on the walls of an artery or vein. With each pulse of the heart, the pressure on the veins and arteries changes in accordance with pressure changes in the blood due to the pumping. Hydrostatic methods may be used to determine blood pressure. Blood pressure measurement using hydrostatic methods, however, may be imprecise.

### SUMMARY

A blood pressure tonometer seeks to measure the pressure waveform in a vein or artery. Noninvasive tonometric measurements based on the arterial blood pressure acting on the arterial wall of an artery or vein, however, may be imprecise due to misalignment, improper applanation, poor calibration, and pre-stress. Pre-stress arises because measurement of the pressure waveform requires the device be pressed against the skin and the achievement of applanation of a vein or artery. In order to contact the skin, applanate a vein or artery and otherwise make measurements of blood pressure, it is necessary to exert a force in the form of pressure on the skin. This initial force or stress, known as “pre-stress,” or “tissue pressure” or “contact based stress” is a source of error in the measurement of tonometric systems. Thus, removing the pre-stress from the measurement by aligning, applanating and calibrating the system may provide more accurate measurement of blood pressure both snapshot and real time.

In one embodiment, a blood pressure system undergoes an alignment, applanation and calibration process to compensate for pre-stress associated with coupling a blood pressure system with an artery or vein. Further, real time measurement may be made possible by properly aligning, applanating and calibration. Pre-stress arises because contact is made by a measurement device with the skin. The pre-stress may be large because it may be necessary to applanate the artery or vein the device is trying to measure. The pre-stress may lead to errors based on drift and the like. This pre-stress may be calibrated out of the system using a calibration device and/or process.

In an embodiment, a blood pressure system may be aligned to a vein or artery. For example, a transducer in a blood pressure sensor may provide an output that may be measured or recorded, for example, by a computing device. Values may be determined form the output, such as, for example, a waveform as measured by a transducer corresponding to a pulse in the artery. The waveform, as measured by a transducer corresponding a pulse in the artery, may vary in relation to alignment. In an embodiment, if the transducer is too far from the artery, no waveform will be seen. As the transducer nears the artery, an output corresponding to the pulse in the artery may be seen. The shape of the waveform can depend on the distance from the transducer to the vein/artery as well as the alignment of the transducer to the vein or artery. Accordingly, comparing the output waveform to one or more predetermined waveform shapes may provide an indication of distance from the transducer to the artery and the alignment of the transducer and the artery. In one embodiment, the transducer may be moved by manual or mechanical means to align the transducer, while in another embodiment, a plurality of transducers, each associated with a spring, load sensor and the like, may be in a single blood pressure sensor, and the transducer with the best wave form may be selected for later applanation, calibration and measurement.

In another embodiment, applanation of a vein or artery may be optimized, such that the amplitude of arterial blood pressure pulsation on the transducer due to each pulse in a vein or artery are maximized. For example, in one embodiment, applanation of a vein or artery may increase the surface area of the vein or artery in contact with a sensor area. As the contact increases, the contrast in pressure from the trough to the peak pressure of a pulse may increase, providing a stronger signal of a pulse. As such, an actuator may actuate a suspended member having a sensor area integrated therein, wherein the sensor area is coupled to a transducer. The first output of the transducer may be determined and the actuator may actuate the suspended member and sensor area. A second output may be determined. The first output and the second output may be compared. Based on this comparison, the actuator may again displace the suspended member and the sensor area.

As an additional example, a frequency spectrum output from the blood pressure sensory may be optimized based on the applanation associated with a vein or artery. For example, the frequency spectra output of a sensor may have several peaks. The relative intensity of the peaks may shift as a vein/artery is more or less applanated. Accordingly, it may be possible to determine the proper point of applanation based on the frequency spectra output of a sensor.

As one example of the embodiment above, the applanation optimization may be performed in conjunction with one or more sensors and one or more computing elements. For example, a computer readable storage medium may be coupled to a processor and the computer readable storage medium may have instructions stored thereon that cause the processor to receive information from the one or more sensors, perform one or more calculations based on the information from the sensors and actuate the system. The frequency spectra output may be compared with predetermined information or other previously recorded data and thus optimized.

In an embodiment, the pre-stress may be calibrated out of the system by calculating the amount of contact based stress caused by pressing a suspended member against a contact surface in communication with an artery or vein. The suspended member may be coupled to a reference by a resilient member having a spring constant. In such an embodiment, the pre-stress caused by the coupling the suspended member with the artery or vein may result in a displacement of the suspended member. The displacement of the suspended member may cause a load to be placed on the resilient member. The amount of surface pre-stress on the vein or artery may be correlated with the load placed on the resilient member having a spring constant, and this value may be used as value for calibration of a noninvasive tonometric pressure measurement. In an embodiment, the load sensor and the transducer may be co-located such that they are receiving information about pressure from the same location.

In one embodiment, the arterial blood pressure acting on the arterial wall of an artery or vein may be determined. As one example, in order to determine the arterial blood pressure action on the arterial wall of an artery or vein, in may be necessary to applanate the artery or vein. The contact based pressure necessary to applanate the artery or vein may be determined by measuring the deflection of a resilient member having a spring constant that couples a reference to a suspended member. The suspended member may also comprise a sensor area, where the sensor area is mechanically coupled to a transducer such that the sensor area transmits an indication of the pressure in an artery or vein to the transducer. The contact based pressure is then correlated with the output of the transducer and the measurement from the transducer is calibrated using the contact based pressure. In such an embodiment, the accuracy of a noninvasive tonometric measurement may be improved.

In another embodiment, the suspended member coupled to a reference or housing by a resilient member may comprise a sensor area coupled to a transducer. As one example, the suspended member and/or sensor area may applanate a vein or artery. The contact based stress caused by applanation may be calculated by measuring deflection of the resilient member and/or by measuring the load placed on the resilient member. In addition, pressure changes in a vein or artery may be transmitted from the sensor area to the transducer and output as an electrical signal. The electrical signal output may be correlated with the contact based stress caused by the applanation process. The system may be calibrated by adding, subtracting or otherwise manipulating the correlated outputs. In addition, the load sensor and the transducer may be collocated such that the load sensor is measuring the pre-stress and other loads on the transducer at the same location as the transducer.

Further to the example above, a calibration process may comprise applanating an artery or vein by actuating a suspended member and determining the transducer peak to peak output and the mean load sensor output under various loads applied by, for example, an actuator. In such an embodiment, a graphical representation of the peak to peak output vs. the pressure may have a shape correlated to and indicative of the systolic and diastolic blood pressure values. Accordingly, the sensor may be calibrated by actuating the suspended member through a series of stresses, and then using the calibrated measurements to conduct real time monitoring.

In an embodiment, a blood pressure sensor may be aligned/positioned based on the waveform of the pulse as measured by the transducer, and then it may be adjusted until applanation is proper based on the frequency output of one or more sensors. After alignment, positioning, and applanation, the blood pressure system may be calibrated either by stepping the sensor through a variety of stresses and watching the wave form, or by removal of the pre-stress. Finally, real time monitoring of the blood pressure may take place with the aligned, applanated and calibrated system.

### BRIEF DESCRIPTION OF THE DRAWINGS

FIG. 1 is a depiction of a wrist mounted blood pressure sensor.

FIG. 2 is a depiction of one example of a system used in the calibration of a blood pressure sensor.

FIG. 3 is a depiction of a transducer and sensor area integral with a suspended member.

FIGS. 4(a)-(c) depict a series of applanation steps in the optimization procedure for a system for measuring blood pressure.

FIGS. 5(a)-(b) depict sample frequency outputs from a sensor applied to an artery or vein for two applanation conditions.

FIGS. 6(a)-(c) depict actuating an actuator to applanate a vein or artery.

FIG. 7 depicts a sample output graph that can be used for measuring blood pressure and calibrating a blood pressure sensor.

FIG. 8 depicts an example method for calibrating a blood pressure sensor.

FIG. 9 depicts an example method for an applanation procedure to optimize applanation for a blood pressure sensor.

FIG. 10 depicts a diagram for the suspended member, pin and transducer in an example alignment configuration with an artery or vein.

FIGS. 11(a)-(e) depict several example output waveforms based on the alignment of the suspended member, load sensor and transducer to the vein or artery.

FIG. 12 depicts an example method for alignment of a blood pressure sensor.

FIG. 13 depicts an example method for determining and optimizing applanation of a vein or artery.

FIG. 14 depicts an additional example method for calibrating and measuring blood pressure.

FIGS. 15(a)-(b) depict experimental results for measuring pressure without and then with calibration by removal of the contact based stress.

### DETAILED DESCRIPTION OF ILLUSTRATIVE EMBODIMENTS

A noninvasive tonometric measurement device comprising a transducer may have error introduced when the transducer is placed in mechanical communication with a vein or artery. This error may be based on improper alignment, improper applanation, pre-stress and/or improper calibration of the system. Isolating and removing these errors from the noninvasive tonometric blood pressure system and/or a blood pressure measurement may increase the accuracy of the measurement.

Proper alignment, applanation, and calibration may lead to accurate real time measurements for a tonometric blood pressure sensor. By examining pulse waveforms, frequency spectra outputs, load on a load sensor, and ratios of transducer output to load sensor output over a range of stresses, a blood pressure sensor may be aligned, properly applanated, and calibrated. Accordingly, a blood pressure sensor can achieve accurate point in time measurements, as well as accurate real time monitoring of the blood pressure.

In one embodiment, a blood pressure measuring device is put in contact with the skin of a user. The device presses against the skin and may also compresses the vein or artery of the user. The compression of a vein or artery is known as applanation of the vein or artery. A waveform based on the pressure in the vein or artery may provide an indication of the alignment of the blood pressure sensor with respect to the vein or artery. Accordingly, correction in alignment may be made, or, in another embodiment where a blood pressure sensor contains a plurality of transducers, the transducer that is most closely aligned may be used.

A process of applanation improves the signal that may be detected from each heartbeat or pulse in a vein or artery and may also improve the measurement of the arterial blood pressure acting on the arterial wall. Further, proper applanation can output a frequency spectra that indicates the amount of applanation. Accordingly, applanation can be set based on the signal strength and/or the frequency spectra output of the transducer. It is important to note, however, that contacting the skin and applanation of the vein or artery introduces an error, which is the pressure necessary to applanate the vein or artery. As such, a system to account for/calibrate out the pressure necessary to applanate the vein or artery can calibrate the device and may improve the reading of the heartbeat or pulse, which can provide a more accurate reading of blood pressure.

In an example embodiment a spring that is suspending an arterial blood pressure pulsation measuring device is loaded by the pressure necessary to applanate a vein or artery. A measuring device measures the load on the spring and a computer correlates the load with the pressure necessary to applanate the vein or artery. The computer also receives the output from a transducer configured to measure the arterial blood pressure acting on an arterial wall, and/or a waveform of a heartbeat from the applanated vein or artery. The computer correlates and calibrates the output of the transducer based on the pressure necessary to applanate the vein or artery.

In another example calibration, a suspended member, load sensor, spring, and transducer are actuated through a series of contact based pressures. At each actuation point, the outputs of the transducer and the load sensor are measured. Peak to peak transducer output values and the mean pressure from the load sensor may be measured and plotted, recorded, used and the like. A graph, table, database, representation, and the like of the relationship between peak to peak transducer output and load sensor mean output may be created. The graph, table, database, representation, and the like may be used to calibrate the device as the graph, table, database, representation, and the like includes is an indication of diastolic pressure and systolic pressure based on the correlation of the two pressures at various pressures applied, for example, by an actuator.

In one embodiment, a noninvasive tonometric blood pressure measurement system comprises: a reference; a suspended member coupled to the reference by a resilient member having a spring constant, wherein displacement of the suspended member gives rise to a load on the resilient member having a spring constant; a load sensor configured to determine the load on the resilient member having a spring constant; and a processor comprising a computer readable storage medium having stored thereon logic configured to correlate the load on the resilient member having a spring constant to a contact based stress causing the displacement of the suspended member.

In another embodiment, a noninvasive tonometric blood pressure measurement system may further comprise a sensor area coupled to a transducer wherein the transducer is configured to output a signal correlated with a pulse incident on the sensor area. In addition, the sensor area may be integral with the suspended member, and, in a further embodiment, the sensor area may be of a different material than the suspended member. As a further example, the computer readable storage medium may have stored thereon logic configured to remove the contact based stress from the pulse incident on the sensor area.

In another embodiment, the noninvasive tonometric blood pressure measurement system may comprise an actuator in communication with the resilient member having a spring constant, the actuator configured to place a second load on the resilient member having a spring constant in a direction substantially perpendicular to the surface contact of the sensor area, and the computer readable storage medium may have stored thereon logic configured to actuate the resilient member having a spring constant. The logic may, for example, be configured to determine the applanation of the fluid conduit comprises logic to measure a first output of the transducer; actuate the resilient member having a spring constant; measure a second output of the transducer; and compare the first output with the second output. As a further example, the logic may be configured to actuate the resilient member having a spring constant based on the applanation of the fluid conduit.

Another embodiment includes a method for calibrating a noninvasive tonometric blood pressure sensor comprising: receiving a first signal from a transducer coupled to a sensor area of a suspended member, which is suspended with respect to a reference by reason of a resilient member having a spring constant; receiving a second signal from a load sensor configured to measure the load on the resilient member having a spring constant; determining a first stress from the first signal; determining a second stress from the second signal; and adding the second stress from the first stress. In a further embodiment, the load on the resilient member has a spring constant that is correlated with a displacement of the suspended member and displacement of the suspended member is caused by contacting the noninvasive tonometric blood pressure sensor with an object.

In an embodiment, the sensor area is coupled to the suspended member, and the first signal is correlated with a pulse incident on the sensor area. As another example, the suspended member may be integral with the sensor area. In another embodiment, the suspended member is configured to displace in a direction perpendicular to a surface contact surface of the sensor area.

In an embodiment, the method above may further comprise determining an applanation value, where determining an applanation value comprises: determining a first pressure value with said sensor area; actuating the suspended member with an actuator; determining a second pressure value with said sensor area; and comparing the first pressure value with the second pressure value. The method may also comprise selecting the actuation of the suspended member based on the applanation value.

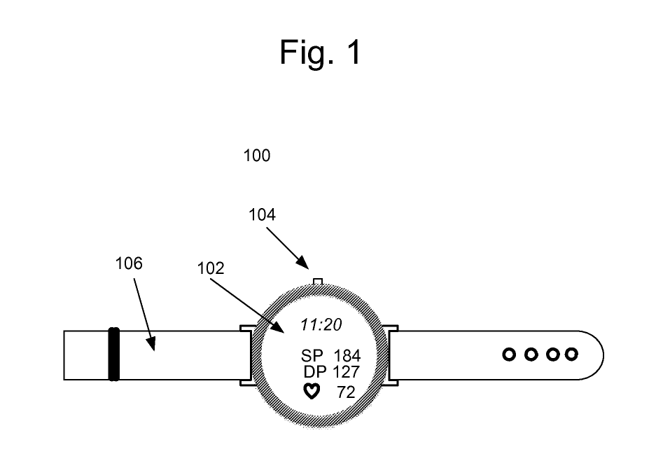

FIG. 1 depicts an example of a noninvasive tonometric blood pressure sensor 100 where a blood pressure system is configured as a wristwatch. The blood pressure system 100 can be used to measure and/or monitor information about a wearer, such as, for example, the blood pressure, heart rate, temperature and the like. Information about the wearer of the watch can be displayed to the wearer, or it may be provided to a third party. The blood pressure system 100 can also comprise other elements, such as, for example, a time piece, a wireless connection, power source, a calendar, or the like.

In one embodiment, the blood pressure system 100 can comprise a single platform, such as, for example a wristwatch. In such an embodiment, aspects of the user such as the blood pressure, temperature, heart rate, and the like can be determined from one or more contacts between the blood pressure system and the user. According to another embodiment, the blood pressure system 100 can be configured as a single unit attached to any body part, such as, for example, a wrist, a finger, arm, leg, chest, head foot, ankle toe or the like.

Elements of the blood pressure system 100 can be configured as one or more platforms attached to a user. For example, the blood pressure system 100 can comprise a watch and a ring. Blood pressure system 100 can comprise, without limitation, one or more of a ring, wrist mount, armband, ankle mount, leg mount, neck mount or the like.

Blood pressure system 100 can also be configured as two or more platforms, wherein a first platform is attached to a user, and a second platform is not attached to a user. For example, the blood pressure system 100 may be configured as a wristwatch attached via a wireless or wired connection to a phone or other portable electronic device, a home computer, a satellite receiver, a communications tower, a network, a server, or the like.

As shown in FIG. 1, the blood pressure system 100 can be configured as a wristwatch and can include a display 102. In one embodiment, blood pressure system 100 measures one or more of the blood pressure, arterial blood pressure acting on an arterial wall, amplitudes of arterial blood pressure pulsation, blood pressure waveforms, heart rate, temperature, and the like and provides this information to one or more elements of the blood pressure system, such as, for example, display 102. In one embodiment, the display 102 is a display that provides information, such as, for example, current blood pressure from blood pressure system 100. As another example, the display may provide a user with information from other elements of the blood pressure system or from outside sources, such as a wired or wireless connection, a clock and the like.

Noninvasive tonometric blood pressure system can also perform the analysis of arterial blood pressure pulsation that can be utilized to advance cardiac diagnostic as well treatment capabilities. In one embodiment, the arterial blood pressure acting on the arterial wall can be used to obtain information about the properties of a fluid flowing in a conduit such as an artery or vein. For example, waveform, measured over time may be correlated with the blood pressure in an artery or vein. Using the amplitudes of the arterial blood pressure pulsation, and/or the arterial blood pressure acting on the arterial wall, elements of a patient health may be monitored. For example, events like incoming heart attack, aortic aneurysm or the response of the watch user to the specific drug can be monitored. The continuous monitoring of the blood pressure, along with other aspects of an individual such as temperature, heart rate and the like can allow for personalization of medical treatment.

Arterial pressure acting on an arterial wall and waveform amplitudes of arterial blood pressure pulsation analysis taken over specific periods of recording times can be used to diagnose other non-cardiac medical conditions like liver, kidney, bladder, and other organs and physiological elements and systematic conditions.

Analysis of the amplitudes of arterial blood pressure pulsation waveforms can be used to monitor treatment, optimize the dose of medications, and check a patient responsiveness to the specific drug and treatment procedures. In addition, the noninvasive tonometric blood pressure sensor can be used to warn a user of coming health problems like arrhythmia, heart attack, valve regurgitation or kidney failure.

The display 102 may comprise any type of display known in the art, for example, without limitation, a digital display, a plasma screen, a touch screen, an LCD screen, a liquid crystal display, a clock face with hands or the like.

The display can be configured to provide the user with several pieces of information at a single time, such as, for example, time, pressure waveform, heart rate, temperature, current blood pressure, blood pressure trends, health status, alerts, medication requirements, time, date, and the like. According to another embodiment, the display may be configurable in any fashion. For example, an input 104 can be pressed or turned or otherwise activated to cause the display 102 to change appearance, show only one piece of information, show multiple pieces of information, and the like.

The blood pressure system 100 can contain any number of inputs 104 in any configuration that can be used as an input to alter one or more characteristics of the display or blood pressure system 100. For example, input 104 can be on the face of the sensor, on the back or side of the system. The input 104 may be configured as a switch, a knob, a keyboard or keypad, a touch pad, a touch screen or the like. In another embodiment, the blood pressure system 100 can comprise a receiver element that can allow for remote input. Input 104 may comprise a remote input element, such as, for example, input from a remote computing environment, input from a portable electronic device or the like.

According to one embodiment, the display 102 may provide a user with other information, such as, for example alignment information. As one example with the blood pressure system 100 configured as a wristwatch, the display 102 may provide alignment information in the form of an arrow, directions to tighten, move left, right, up or down on the arm or the like.

The blood pressure system 100 can have a startup or default mode of operation. The blood pressure system 100 may be configurable at startup or during operation in any fashion using inputs 104 as described above.

The noninvasive tonometric blood pressure system 100 can comprise an element to attach the blood pressure system 100 proximate to a vein or artery. The attachment element can comprise a strap 106, harness, clamp, adhesive, elastic band, a leather watch band, metal links or any other type of strap, harness or the like can be used to affix the sensor. In one embodiment, the blood pressure system 100 is attached to a user by an attachment element such as, for example, strap 106 such that a transducer element on blood pressure system 100 is in proximity to a vein or artery.

The strap can be integrated with various auxiliary sensors such a temperature, stress, strain, humidity, motion monitoring, sensors and the like, that provide additional information and data that can enhance the measurement capabilities of the watch as well improve the measurement procedure, accuracy and reproducibility of the watch as well to provide the input to the user when the expected measurement conditions are present.

The blood pressure system 100 may be mounted on a human. In another embodiment, a noninvasive sensor can be mounted on an animal, such as, for example, a horse or a dog.

The blood pressure system 100 may be mounted on any pulsatile fluidic system (liquids, gases) in various industrial, environmental, etc. systems for the purpose of testing, process control, safety, fluidic system operational conditions in vehicle, etc.

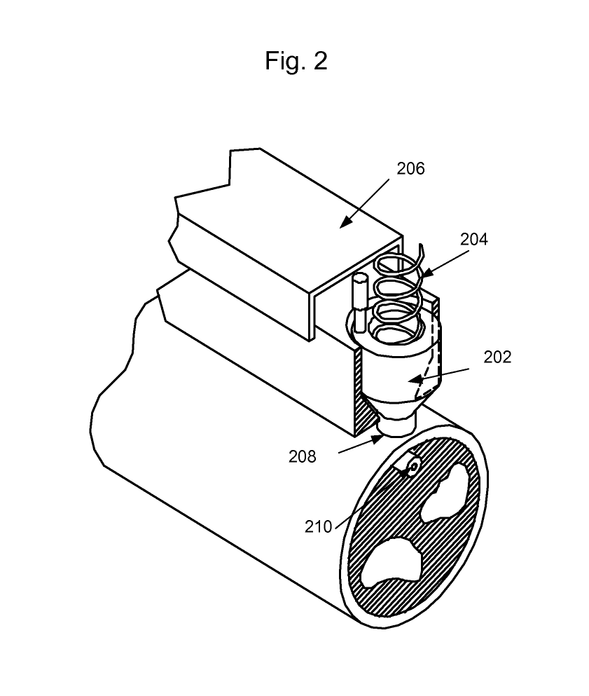

FIG. 2 depicts a system for calibrating a noninvasive tonometric system. In a first embodiment, the system for calibrating a blood pressure measurement system may comprise a wrist watch 100, or may otherwise be mounted on a human in one or more of manners such as those described above with respect to FIG. 1.

In an embodiment, the system for calibrating a noninvasive tonometric measurement system may comprise a suspended member 202. The suspended member may be made of any material suitable for contacting one or more surfaces and being suspended by a resilient member having a spring constant 204. For example, the suspended member may be metallic, plastics, glass, a composite, wood, stone, rubber, or any other suitable material. In one embodiment, the suspended member 202 may be made of a single material, while in another embodiment, it may comprise two or more materials. The suspended member 202 may be of any shape known in the art. For example, it may comprise one or more of a cube, a sphere, a cylinder, a cone, a tube, a block, a pyramid, an amorphous shape, an angled block, or any other shape known in the art. As a first example, each of these shapes may be solid. As another example, the shapes may be hollow or have one or more cavities associated therewith.

The suspended member 202 may be coupled to a resilient member having a spring constant 204. In a first embodiment, the two may be coupled directly, however, one or more measuring units, layers of material, springs or any other suitable attachment means may be situated between the suspended member 202 and the resilient member having a spring constant 204. The resilient member having a spring constant 204 may be a cantilever spring, a coil spring, helical spring, volute spring, balance spring, leaf spring, V-spring, Belleville spring, rubber band, wave spring, gas spring, any elastic material or any other suitable resilient member having a spring constant. The resilient member may be made of any suitable material know in the art. The spring 204 may couple the suspended member 202 to a reference 206, and, in an embodiment, a load may be placed on the resilient member having a spring constant 204 when the suspended member 202 undergoes a deflection.

The suspended member 202 may be attached to a reference 206. The reference 206 may be attached to the suspended member 202 by any resilient member having a spring constant 204 known in the art. The reference 206 may comprise any solid material known in the art and may be of any shape or configuration suitable for creating a reference for the attachment of a resilient member having a spring constant 204.

In FIG. 2, the reference is shown as being directly attached to the resilient member having a spring constant 204, however, in one embodiment one or more layers of material, or one or more devices may be situated between the resilient member having a spring constant and the reference. For example, a second resilient member having a spring constant may couple the first resilient member having a spring constant 204 to the reference 206. In addition, a load sensor may be situated between the resilient member having a spring constant 204 and the reference 206. In one embodiment, a load sensor associated with the resilient member having a spring constant 204 may measure the load on the resilient member having a spring constant.

The suspended member 202 may be placed in communication with a vein or artery 210 by contacting the suspended member at a surface 208. In one embodiment, the surface is skin. In another embodiment, the surface 208 may be any surface capable of communicating mechanical information about pressure in a pulsatile system to a transducer.

In one embodiment, contacting the suspended member 202 at the surface 208 may exert a force on the suspended member, causing displacement of the suspended member, 202, which may place a load on the resilient member having a spring constant 204. This load may be expressed as, in one example, the compression of a compression spring. In one embodiment, the displacement of the suspended member and the deflection of the resilient member may be in a direction substantially perpendicular to the surface contact between the suspended member and the skin. In another embodiment, the resilient member may be configured to deflect at some angle from perpendicular from the surface contact of the suspended member and the skin. It is envisioned that a deflection of the resilient member may be determined at nearly any angle and may be configured in any appropriate way.

In FIG. 2 the surface contact 208 is situated near a vein or artery 210. This vein or artery 210 may have blood running there through and each heart beat may cause a pulse in the vein or artery 210. The surface 208 may transmit mechanical information related to the pressure in the vein or artery to the suspended member 202. As one example, a pulse from a heart beat may cause a force to be exerted through the surface 208 on the suspended member 202. As an extension of this example, the amount of force received by the suspended member may be proportional to a pressure exerted on the contact 208 by the suspended member 202. As noted above, this pressure exerted on the contact 208 may cause deflection of the resilient member having a spring constant 204.

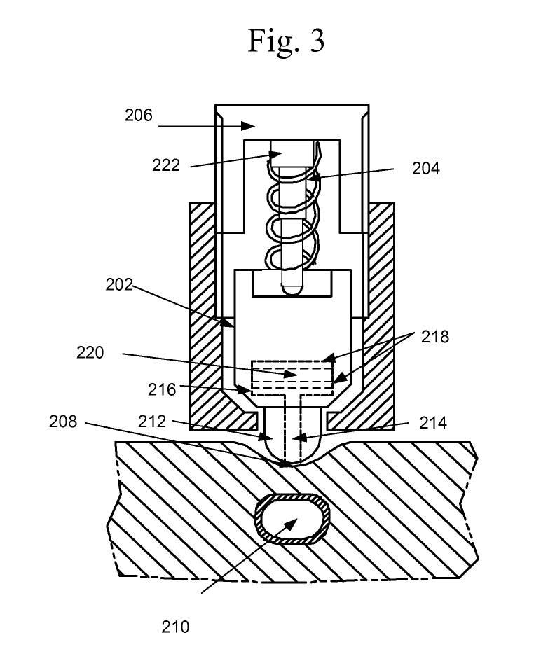

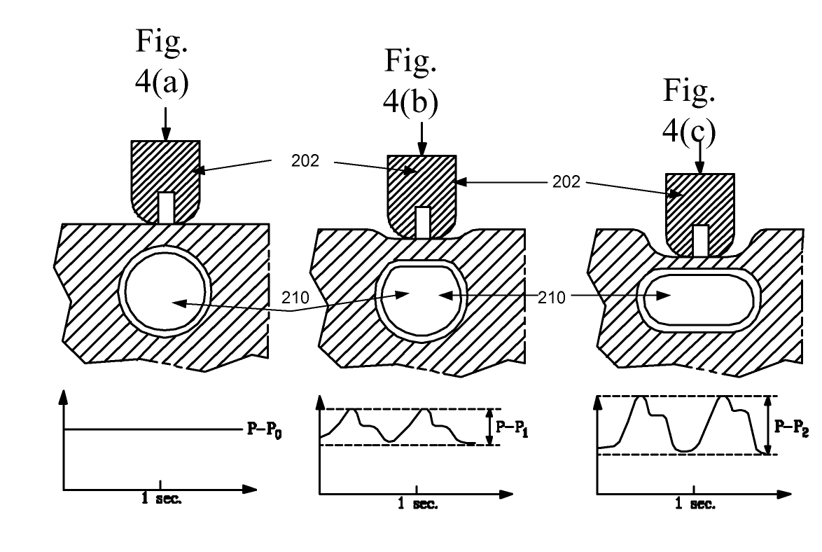

FIG. 3 is an embodiment of the pressure sensor of FIG. 2 above, and may include each element described above, including but not limited to, the spring 204, the suspended member 202, the reference 206 and the contact surface 208.

In an embodiment, as shown in FIG. 3, the suspended member 202 may comprise a protrusion 212. The protrusion 212 may be a hemisphere, cylinder, ellipse, elongated dome, dome, tube, pyramid, cone, cube, block, amorphous and/or any other shape known in the art. Each shape may be hollow or solid. The protrusion 212 may comprise one or more layers of any solid material and may be contacted with a surface at 208. In one embodiment, the protrusion 212 may be ergonomically shaped and may be configured to be situated between the tendons of a human's wrist.

In an embodiment, the protrusion 212 may transmit an indication of pressure from contacting the calibration system 200 with, for example, skin, to the resilient member having a spring constant 204. The indication of pressure may place a load on the resilient member having a spring constant 204 and may cause a deflection of 204.

The suspended member 202 may also comprise a pin 214. The pin 214 may be of any material suitable to send an indication of pressure in a vein or artery to a transducer 220. The pin may be any solid or semi-solid such as a metal, plastic, glass, or the like. The pin may be flush with the protrusion 212 at the surface contact 208, or it may extend out past the protrusion 212, or it may be receded inside of the protrusion 212.

In another embodiment, the pin may be integral with the suspended member 202. In such an example, the pin 214 and the sensor area may comprise the same material and may comprise the same space. In another embodiment, the pin 214 may be of the same material but may be situated in contact with the suspended member 202. In a further embodiment, the pin 214 may not be touching the suspended 202 member at all.

In one embodiment, the pin may extend up into the suspended member 202 and may comprise a coupling surface 216 for coupling the pin 214 to the transducer 220. The portions of the pin 214 and 216 may be of any material and may be rectangular, cube, block, rounded, elliptical, cone, spherical, tubes, cylindrical, amorphous or any other shape. The pin may be made of multiple materials or layers and, in one embodiment may comprise one or more electrodes 218.

The tip of the pin 214 contacting at the surface 208 may be considered the sensor area of the suspended head 202, and more generally of the calibration unit of a noninvasive tonometric sensor. The sensor area may contact, for example, the skin of a wearer and may transmit in indication of the pressure in a vein or artery to a transducer 220. The sensor area may comprise the same material as the pin or it may comprise a different material and may be shaped in any way to receive an indication of pressure from, for example, skin and transmit that indication through the pin. The pin 214, suspended head 202, load sensor 222, and transducer 220, may therefore all be co-located in that they are in communication, mechanical or otherwise with the contact surface 208. Accordingly, measurements are related to the same area of the transducer 220 and the load sensor 222.

The suspended member may comprise electrodes 218. These electrodes 218 may receive an electrical signal from a transducer 220 and transmit the output to a computing device (not pictured). They may be of any shape, may comprise multiple materials or layers and may be made from any one or more conducting materials. The electrodes 218 may be charged and may operate in conjunction with, for example, a piezoresistive material used as transducer 220. In another embodiment, they may work in conjunction with a piezoelectric material operating as transducer 220. While the electrodes 218 in FIG. 3 are configured such that one electrode 218 is configured in between the pin 214 and the transducer 220, and the other electrode 218 is configured across the transducer 220, such a configuration is one example only and is not limiting. The electrodes 218 may be on the same side of a transducer 220 or they may be configured on any two surfaces of the transducer 220 such that they may transmit to or receive a signal from transducer 220.

Although FIG. 3 depicts a single suspended member 202, spring 204, load sensor 222, transducer 220, pin 214, etc. and the like, it is important to note that this is just an example configuration. It is conceived that there may be several suspended members, each with associated parts as depicted in FIG. 3. These several suspended members may be in an array, thereby reducing the difficulty in alignment with one or more of the arrays with a vein or artery. Each of the sensors associated with each of several suspended members may be in contact with one or more computers. They may be used in conjunction with each other, or they may be capable of being isolated. Accordingly, it is understood that throughout this document, single suspended members, transducers, load sensors, pins etc. may be replaced by a plurality of each of these elements.

The calibration system may comprise one or more computers. Each computer may have one or more processors, one or more memories, and may be suitable to receive inputs from various devices, including but not limited to user input devices such as keyboards, mice, touchscreens, transducers, sensors, optical devices, audio devices, mechanical measuring units and the like. The computer store those inputs in memory and process them. In addition, the computer may comprise one or more displays which may provide information to a user or to a third party. The computer may be capable of connecting in a wired or wireless fashion to any network or other computing device. As such, any and all programs, data, inputs and/or outputs may be transferred between the computer and another computing device to which it is connected.

The computer may comprise a computer readable storage medium having stored thereon, instructions comprising logic configured to cause a processor to perform various actions. The computer readable storage medium may be removable or not and it may comprise a disk, drive, flash memory, an optical storage medium, or any other computer readable storage medium known by one having skill in the art. The instructions comprising logic may configure a general purpose computing device as a special purpose computing device suitable for one or more functions or operations. As such, a computer may transform input of one type into output of another type, may operate as a general purpose computing device and a special purpose computing device, and may, among other things, provide instructions to one or more devices and/or systems to perform mechanical or electrical functions.

In one embodiment, the computer readable storage medium may have stored thereon, instruction comprising logic configured to optimize the applanation of a vein or artery using outputs from a transducer and by providing instructions to actuate an actuator. As another example, instructions comprising logic configured to determine the blood pressure in a vein or artery using the output from a transducer such as transducer 220 and the output from a load sensor such as load sensor 222. In such an example, the logic may comprise a calibration procedure, various mathematical functions and the like.

The suspended member 202 may comprise a transducer 220. In one embodiment, the transducer 220 may comprise one or more layers wherein mechanical energy may be converted into an electrical signal. For example, the transducer 220 may comprise any piezoelectric material including but not limited to piezoelectric crystals, piezoceramics, piezoelectric biological materials and piezoelectric polymers. In another embodiment, the transducer 220 may comprise one or more layers wherein mechanical energy may change one or more properties of the material. In one example, the resistance of the transducer 220 may change when placed under pressure. In such an example, piezoresistive materials may be used. For example, any piezoresistive metal, semiconductor, or any other piezoresistive may be used as transducer 220.

The transducer 220 may receive an indication of pressure from the sensor area where the indication is correlated pressure in the vein or artery. The transducer 220 may output a signal that may be processed by the computer. The computer may perform one or more actions with the output of the transducer.

As a first example, the computing device may comprise a computer readable storage medium having stored thereon, logic configured to correlate the transducer 220 output with the output from a load measuring device 222. The computing device may perform one or more mathematical functions in order to correlate the two outputs and to combine them in some fashion. For example, the outputs may be added, subtracted, multiplied, divided, or subject to any other mathematical function or algorithm known.

In an embodiment, the computing device may be associated with logic configured to receive information from transducer 220 and load sensor 222 before the suspended member 202 and the transducer 220 have not been brought in contact with, as one example, a wrist. Accordingly, the computing device may be associated with logic configured to record transducer 220 and load sensor 222 information, and to associate this information with values, which may be offset values. As one example, the offset values may be computed by taking the time average of the signal output from the transducer 220 and the time average of the signal output from the load sensor 222.

Further to the example above, the suspended member 202 may be coupled with the contact surface 208. Contact pressure at the contact surface 208 causes a displacement of the suspended member 202 measured in conjunction with the transducer 220. In an embodiment, the displacement of the suspended member 202 places a load on the resilient member having a spring constant 204. The load placed on the resilient member is measured by the load sensor and signals may be transmitted to the computing device. The computing device may receive a series of signals and determine the difference between signal output of the trough and the peak amplitudes and time average of the signal output from the transducer 220 after subtracting the offset and the time average of the signal output from the load sensor 222 after subtracting the offset.

Depicted in FIG. 3 is a load sensor 222. The load sensor 222 measures the load on the spring 204. The load sensor 222 may comprise any device suitable for detecting load on a spring. It may be an optical sensor, a transducer, a scale, a mechanical compression measurement unit or any other device known by those having skill in the art. The load sensor 222 may be configured to transmit information related to the load on the resilient member to any other system or element. For example, the load sensor may transmit an indication of the load on the resilient member to the computing device which may use the information in one or more ways.

As a further example, the computing device may provide information based on the output of the transducer to one or more of a user or any third party. This output may comprise arterial pressure acting on an arterial wall, a blood pressure reading, a waveform, a graph, signal, or the like. In addition, the output from the transducer may first undergo one or more mathematical functions, including correlation and combination with an output from a load sensor 222, before being provided in some fashion to a user or a third party. As a specific example, the transducer output may have the load sensor 222 output added to it, noting that one or more values may be negative, and the result may be correlated with the blood pressure and provided to the user or third party.

In another embodiment, the output of the transducer may also be used in conjunction with an actuator in order to optimize the output of the signal from the transducer. In such an embodiment, an actuator which may vary greatly in form and function may operate to increase or decrease the pressure between the contact surface 208 and the suspended member 202. For example, actuation may be caused by tightening or loosening a wristwatch, or it may be a mechanical or electromechanical system that introduces pressure on the suspended member or the resilient means. By changing the pressure between the contact surface 208 and the suspended member 202, the vein or artery being measured will undergo varying degrees of applanation. By actuating the suspended member 202, the optimum level of applanation may be determined. For example, a first output from a transducer 220 may be processed by a computing device and stored in memory. The computing device may instruct the actuator to increase the force on the surface contact of the suspended member 202. A second output from the transducer 220 may be processed by the computing device and compared with the first output from the transducer 220. Based on the comparison, the computing device may determine if the increase in force was advantageous or not, and thereby whether to continue to increase force, maintain the present level of force, or to decrease the force.

FIG. 4(a)-(c) provides a graphical depiction of the process of applanation on a vein or artery. As shown, in FIG. 4(a), when the suspended member and associated elements are placed just in contact with the surface contact 208 of the skin, no applanation is seen and little or no signal from the heartbeat is read on the graph. In FIG. 4(b), as pressure, which may cause deflection of the resilient member having a spring constant 204 and be measured by the load sensor 222, increases, the vein or artery 210 begins to applanate. A signal may now be measured by transducer 220. As the vein/artery undergoes further applanation in FIG. 4(c), an even better signal is seen, however, there is a limit to this increase in signal as eventually the vein or artery will occlude, or the contact may otherwise become unstable. Thus, finding the optimum level of applanation may be provided by measuring the output of a transducer, actuating the suspended member, measuring a second output, comparing and actuating again until a maximum is found.

In another embodiment, the transducer 220 and load sensor 222 may be associated with a computing device. The computing device may comprise, among other things, one or more processors and one or more memories which may be computer readable media. The computer readable media may have stored thereon instructions, the instructions configured to run on the processor. The instructions may cause the processor to perform one or more steps. Although not pictured in FIG. 4, in one embodiment, the transducer 220 and load sensor 222 may output a signal prior to coming into contact with a contact surface such as contact surface 208. The output of the load sensor 222 and the transducer 220 may be transmitted wired or wirelessly to the computing device and stored in one or more memories. The unloaded values of the load sensor 222 and the transducer 220 may be used as a reference or an offset value.

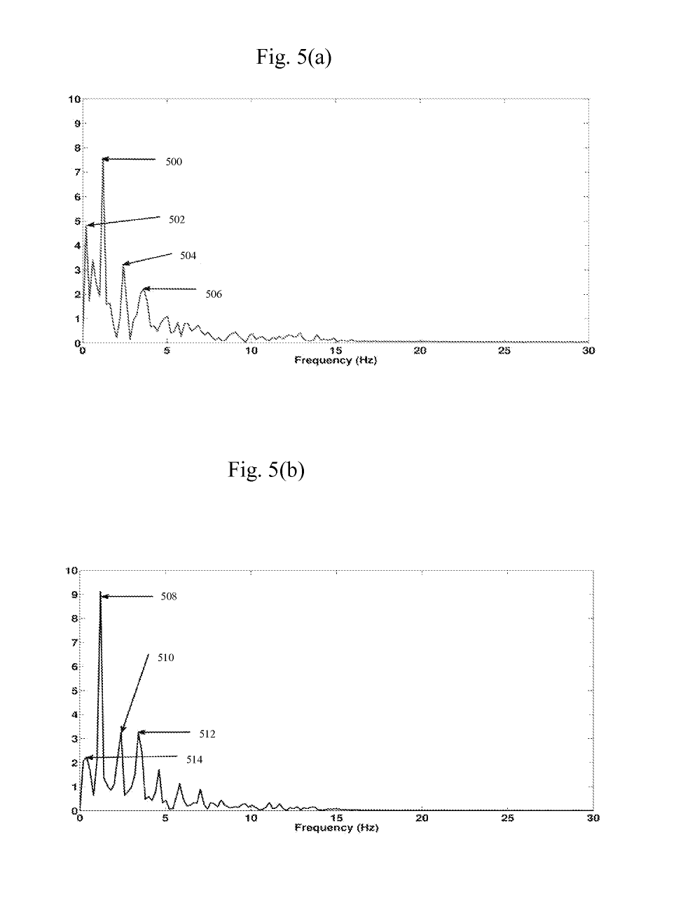

FIGS. 5(a)-(b) depict a frequency spectra output of a sensor, such as transducer 220 of FIG. 3, which may be used to determine the amount of applanation of a vein or artery as depicted above in FIGS. 4(a)-(c). The blood pressure measurement for each applanation point in 4(a)-(c) will have a different frequency spectra associated therewith. This frequency spectra can be detected. FIG. 5(a) depicts the frequency spectra output of transducer 220 when the applanation is at or near ideal. FIG. 5(b) depicts a frequency spectra output of the transducer 220 when the applanation is not ideal. Accordingly, reviewing the relative peaks of the output of transducer 220 may provide an indication of proper applanation.

To obtain the graph in FIG. 5(a), the output of a transducer may be provided to a computing device. As one example, the output may be indicative of correct applanation, such as in FIG. 5(a). Here, several peaks 500, 502, 504, and 506 may be seen. Importantly, peak 504 has a higher amplitude than peak 506. At this condition, the applanation is correct or nearly correct.

To obtain the graph in FIG. 5(b), the output of the blood pressure sensor may be provided to a computing device. The vein or artery, however, is over or under applanated. Accordingly, although several peaks 508, 510, 512, and 514 are seen, it is important to note that 512 and 514 are now equal. Accordingly, the condition where 504 was of a greater amplitude than 506 is not met in FIG. 5 (b), indicating an improper amount of applanation.

There may be means for determining the frequency spectra of the blood pressure sensor and means for comparing the frequency spectra with predetermined spectra, or there may be means for processing the spectra to determine if peak 506/510 has a greater amplitude than peak 508/512. As on example, this may include a computer.

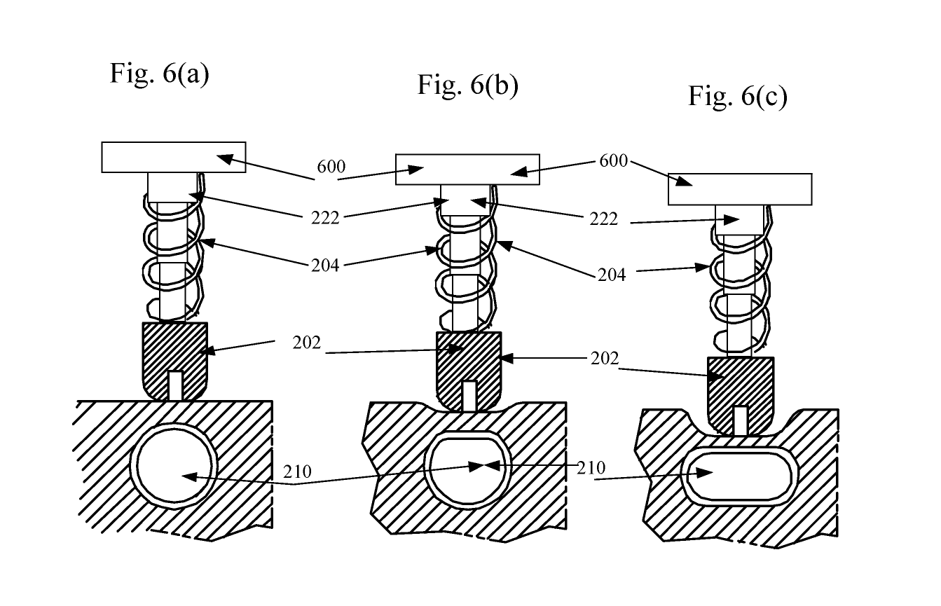

FIGS. 6(a)-6(c) provide a graphical description of applanation of a vein or artery using an actuator 600. FIG. 6(a) depicts, as in FIG. 4(a), a suspended member and associated elements placed just in contact with the surface contact 208 of the skin. In FIG. 6(a), actuator 600 is not applying any pressure to the surface contact 208 of the skin and thus the vein or artery 210 has undergone no applanation. In such an example, the load sensor 222 may determine that no load has been placed on the resilient member having a spring constant 204 correlating with a displacement of the suspended member 202. A signal may be sent from the actuator 600 to the computing device which may include an indication that the actuator is not actuating the resilient member and/or the suspended member. In addition, the load sensor may send a signal to the computing device which may include an indication that the load sensor does not detect a load on the resilient member,

FIG. 6(b) depicts a vein or artery 210 undergoing some applanation, where the vein or artery is not fully flattened but stress has been applied. In FIG. 6(b), the actuator 600 may have received an instruction to actuate from a computing device. Actuator 600 may have actuated such that the vein or artery 210 undergoes applanation. This actuation may have occurred based on an instruction provided by a computing device to the actuator 600. A load may have been placed by the actuator 600 on the resilient member having a spring constant 204 and/or the suspended member 202. The load sensor 222 may determine that the load has been placed on the resilient member having a spring constant 204 and may send an indication correlated with the load to the computing device. In one embodiment, when the actuator places a load on the resilient member 204 and or the suspended member 202, the transducer 220 may output a signal related to the pressure in the vein or artery 210. This output may also be provided to the computing device. In one embodiment, the outputs of the load sensor, the actuator and the transducer 220 may create a feedback loop such that optimization of the signal output of the transducer 220 may be determined. For example, an increase in applanation of a vein or artery 210 may cause in increase in the strength of the signal and/or increase in the amplitude of the waveform of arterial pressure acting on an arterial wall and the transducer 220. This increase may have a maximum and by actuating the actuator 600, calibrating based on the load sensor 222 and measuring the signal from the transducer 220, then actuating again, a maximum may be found.

FIG. 6(c) depicts a maximum in applanation of vein or artery 210. In such an embodiment, the actuator 600 has provided enough of a load on the resilient member having a spring constant 204 and/or the suspended member 202 to fully applanate the vein or artery 210. The maximum may have been found by providing outputs from the load sensor, the actuator 600 and the transducer 220 to a computing device. Instructions may have been provided to the actuator to actuate in one or more ways, such as, for example, increasing or decreasing stress. Thus, an optimal applanation point may be determined.

Actuator 600, may be any actuator known in the art, including manual, electronic, and/or mechanical implementations of an actuator. In one embodiment an actuator may comprise the tightening of a c-clamp, wristband, strap, screw, or the like. Or it may comprise sending an electronic signal to a mechanical actuation device. Any other actuator known in the art may be used to increase or decrease the pressure of the suspended member, the transducer, the load sensor, the resilient member having a spring constant and/or any other elements of FIGS. 1-3 depicted above.

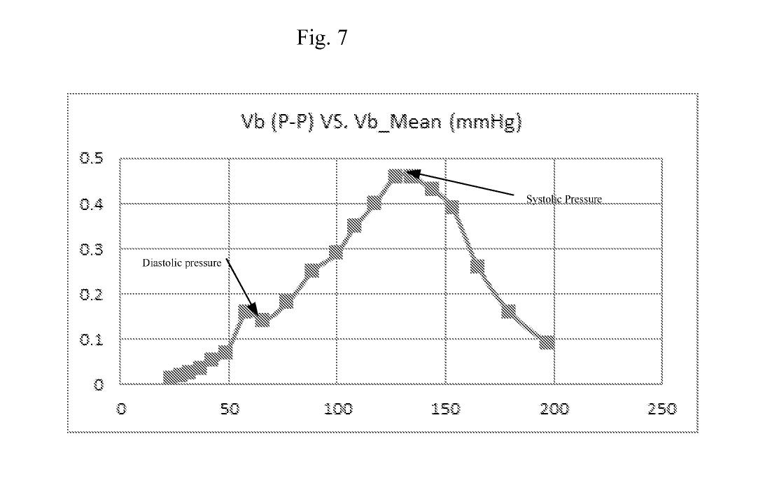

FIG. 7 depicts an output from a transducer such as transducer 220 for a blood pressure system 100. In FIG. 7, two values are plotted. The first value, Vb(P−P) is the peak to peak value output measured from transducer 220. The second value, Vb_mean is the mean value output from the load sensor 222. The number on the left side of the graph is the voltage and the numbers on the bottom are contact pressure between the suspended member and the skin, shown in mm of mercury. As can be seen in the plot, there are two peaks.

As the transducer 220 and load sensor 222 are actuated, the amount of pressure increases and the voltage Vb(P−P) is measured. The graph displays two peaks and a deflection point and/or trough, the deflection and/or trough indicating the diastolic pressure and the right peak indicating the systolic pressure. Accordingly, performing an actuation to increase pressure, measure and then process provides an indication of the systolic and diastolic pressures, which may be used as a measurement, and which may also be used to calibrate the blood pressure sensor for real time and/or snapshot blood pressure measurements.

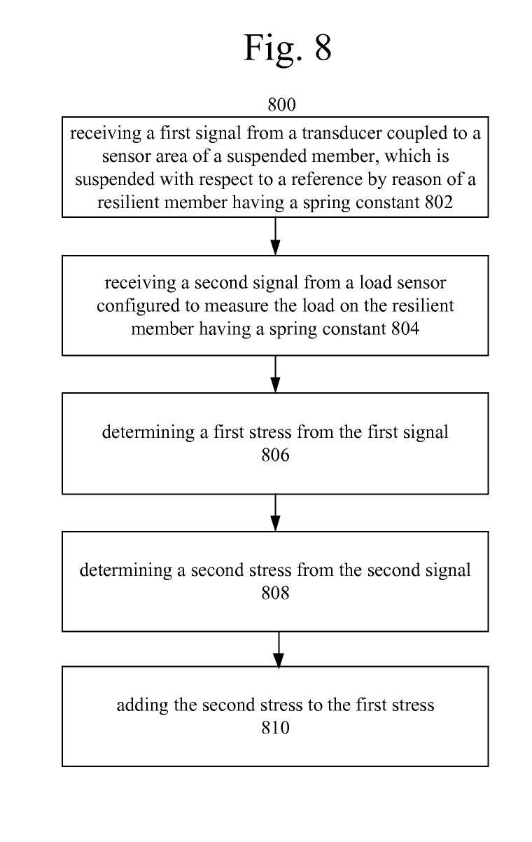

FIG. 8 depicts an embodiment of a sample method for calibrating the contact based stress out of a noninvasive tonometric blood pressure measurement device 800. At 802, a first signal may be received from a transducer coupled to a sensor area of a suspended member, which is suspended with respect to a reference by reason of a resilient member having a spring constant. In an embodiment, the transducer may be the transducer 220 described above with respect to FIG. 3, and the suspended member may be the suspended member 202 described above with respect to FIGS. 2 and 3. Further, the resilient member having a spring constant may be the resilient member having a spring constant described above with respect to FIGS. 2 and 3. In one embodiment, a computer as described above may receive the first signal as an input and may store the signal in memory or process or perform any other function on the first signal. Accordingly, step 802 may comprise means for receiving a first signal from a transducer coupled to a sensor area of a suspended member, the suspended member being suspended by a resilient member having a spring constant.

At 804, a second signal may be received from a load sensor configured to measure the load on the resilient member having a spring constant. In an embodiment, the load sensor may be load sensor 222 described above with respect to FIG. 3. The second signal may be received by the computer as described above and the computer may store the signal in memory or process it or perform any other function on the second signal. Accordingly, step 804 may comprise means for receiving a second signal from a load sensor configured to measure the load on the resilient member having a spring constant.

At 806, a first stress may be determined from the first signal. In an embodiment, the first stress may be indicative of the pressure in a vein or artery. In another embodiment, the first stress may be indicative of a heartbeat or a pulse. In one embodiment, a computer which receives the first signal determines the first stress. Accordingly, step 806 may comprise means for determining a first stress from the first signal.

At 808, a second stress may be determined from a second signal. In an embodiment, the second stress may be indicative of the contact based stress applied by applanating a vein or artery. In another embodiment, it may be the tissue stress. In one embodiment, a computer which receives the second signal determines the second stress. Accordingly, step 808 may comprise means for determining a second stress from the second signal.

At 810, the first stress may be added to the second stress. Although adding is described herein, a negative number may be added to a positive value and vice versa. Accordingly, step 810 may comprise means for adding the second stress to the first stress.

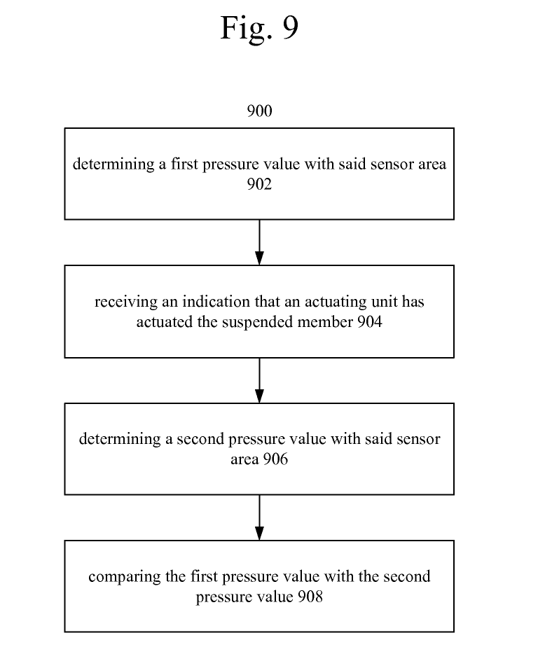

FIG. 9 depicts an example method for optimizing the applanation of a vein or artery 900. At 902, the pressure value may be determined. The first pressure value may be a waveform/transducer output which is correlated with the amplitudes of arterial blood pressure pulsation in a vein/artery during the course of a heartbeat. It may also be a numerical value related to pressure or any other value. The first pressure value may be correlated with an output from a transducer such as transducer 220 described above with respect to FIG. 3. In one embodiment, the computer that receives the first signal determines the first value. Accordingly, step 902 may comprise means for determining a first pressure value with a sensor area.

At 904, the method may comprise receiving an indication that an actuator, such as actuator 600 above has actuated the suspended member. In one embodiment, the initiation and extent of the actuation may be instructed by the computer and the indication may be received from the actuation unit, from the computer or as an output from any other device, such as, for example, the transducer. This indication may comprise an indication that a new reading by the transducer should be taken. Accordingly, step 904 may comprise means for receiving an indication that an actuator has actuated the suspended member.

At 906, a second pressure may be determined with the sensor area. The second pressure value may be a waveform/transducer output which is correlated with the amplitudes of arterial blood pressure pulsation in a vein/artery during the course of a heartbeat. It may also be a numerical value related to pressure or any other value. The second pressure value may be correlated with an output from a transducer such as transducer 220 described above with respect to FIG. 3. In one embodiment, the computer that determines the first value determines the second value. Accordingly, step 906 may comprise means for determining a second pressure value with the sensor area.

At 908, the first pressure value is compared with the second pressure value. In one embodiment, the values are compared by the computer. In addition, further actuation in the same direction as the previous action may take place based on the comparison, or, in another embodiment, actuation in the opposite direction may take place based on the comparison. In a further embodiment, based on the comparison, no more actuation may take place. Accordingly, step 908 may comprise means for comparing the first pressure value with the second pressure value.

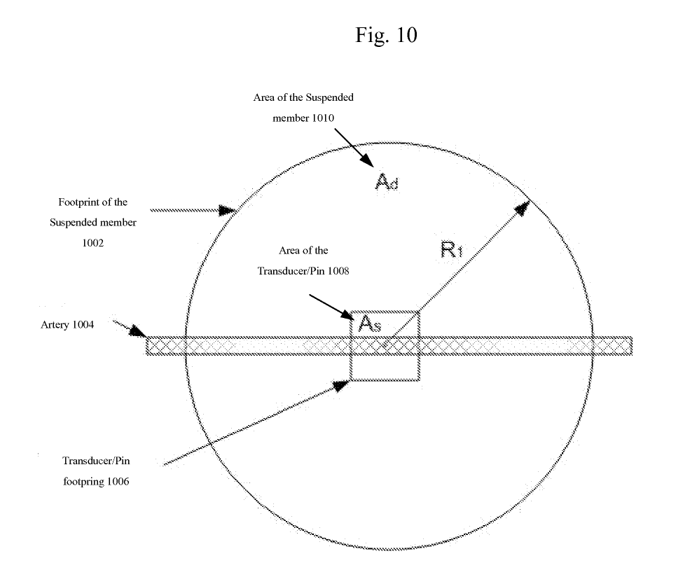

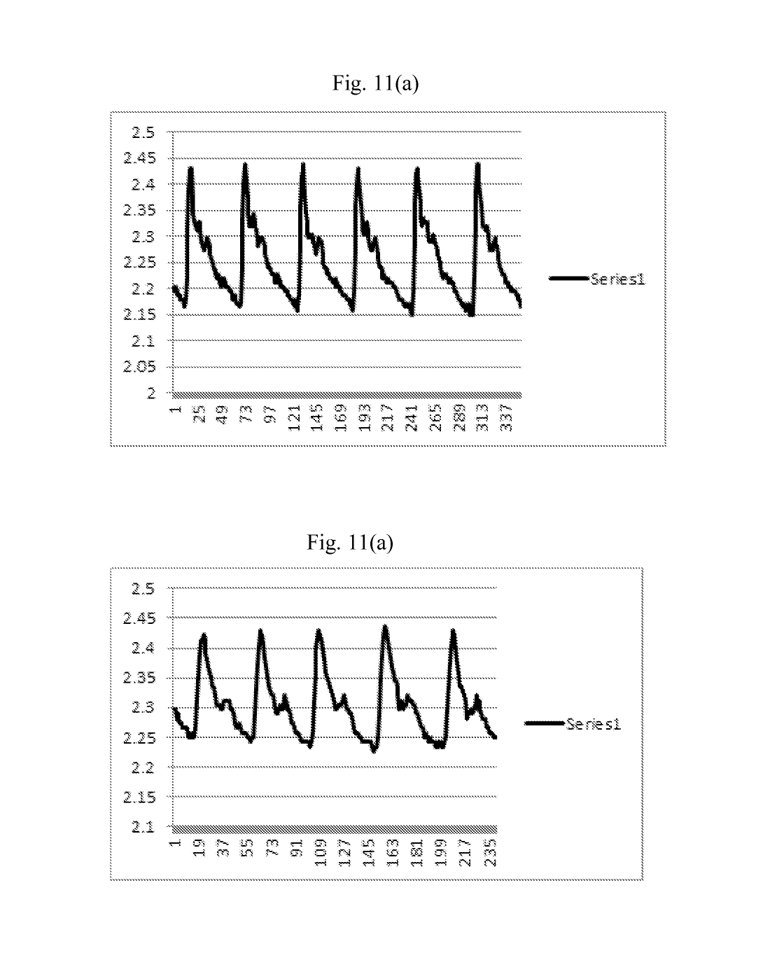

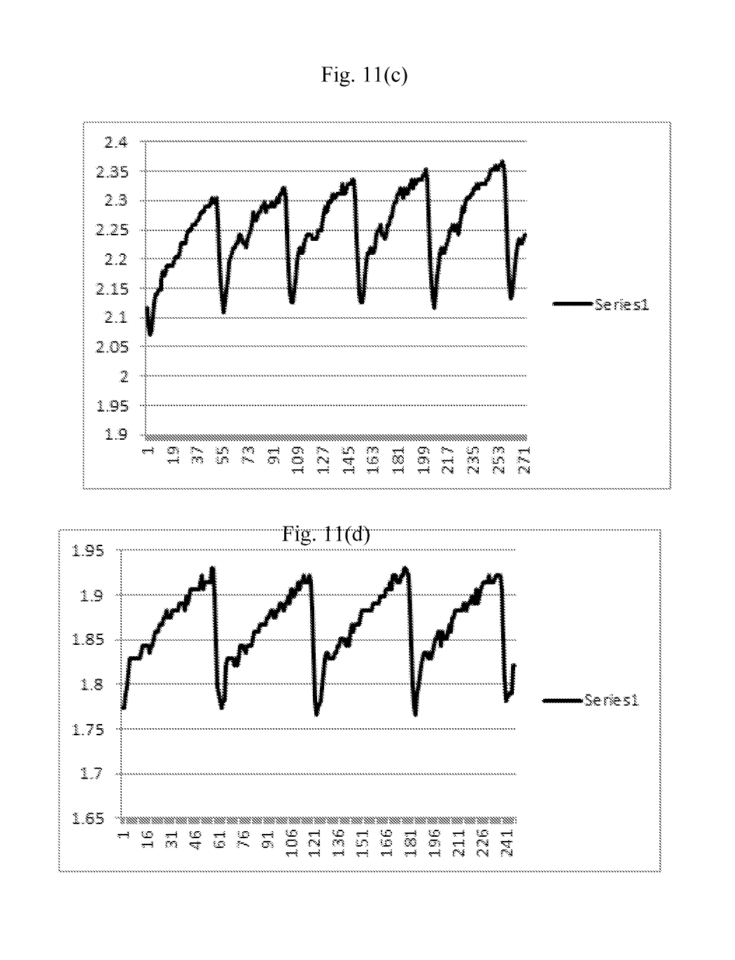

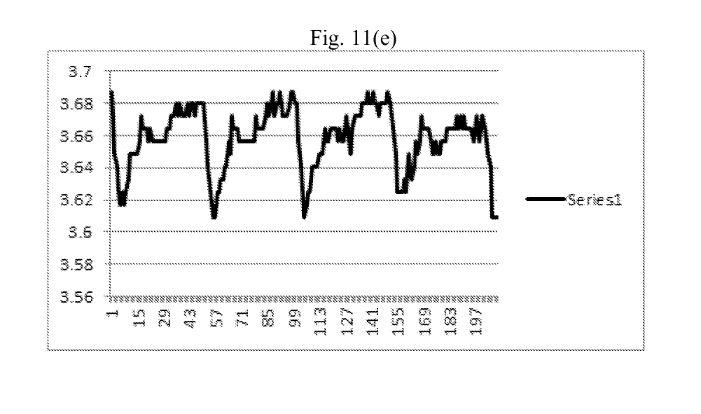

FIG. 10 depicts an optimized alignment of a transducer 220 to an artery or vein. The outer circle represents the footprint of the suspended member 1002, which may be associated with an area of the suspended member 1010 Ad. The footprint of the transducer 1006 may be associated with the area of the transducer/pin 1008. In FIG. 10, the alignment of the artery and the transducer is ideal. Ideal alignment leads to the proper waveform, as depicted below in FIG. 11.

FIGS. 11(a)-(e) depicts a series of output waveforms from the load sensor and the transducer. FIG. 11(a) is the ideal waveform, which will be seen when the transducer and the artery/vein 210 are aligned. FIG. 11 (b) depicts a sample waveform when the sensor is placed approximately 1 mm to one side or the other of the artery or vein such as artery 1004. FIG. 11 (c) depicts the waveform at 2 mm to the side and 11(d) at 3 mm. 11 (e) depicts the waveform at 4 mm and finally, little or no waveform was found at or beyond 5 mm. It can be seen that the waveform changes significantly, appearing to ‘flip’ upside down as the distance from ideal changes. The amplitude of the waveform can be similar in magnitude at a distance, but the waveform changes. Accordingly, the waveform may be detected and a computing device may store the waveform. The waveform may be compared with a predetermined waveform by a computing device, or otherwise used to determine the quality of the alignment of the transducer. Alignment of the transducer may provide more accurate results.

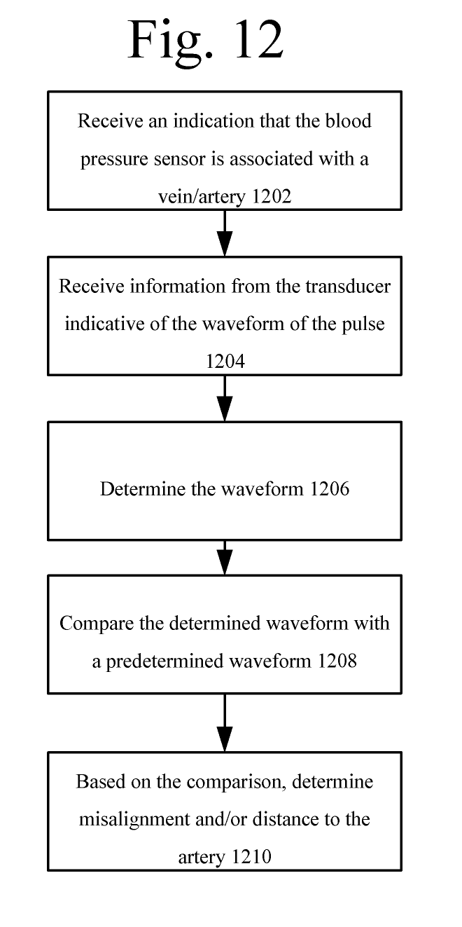

FIG. 12 depicts an additional example method for calibrating and measuring blood pressure. At step 1202, an indication may be received that the blood pressure sensor is associated with a vein or artery. The indication may be from a sensor, such as the transducer 220 or load sensor 222, or otherwise be an indication from the blood pressure sensor. A means for receiving an indication that the blood pressure sensor is associated with a vein or artery may also be included.

At step 1204, information may be received from the transducer, and the information may be indicative of the waveform of the pulse. As one example, the output of the transducer may be a voltage, a current, or an electrical signal. The information may or may not be converted by a converter and it may be transmitted by any means of wired or wireless transmission known in the art. Accordingly step 1204 may comprise means for receiving information from the transducer, the information indicative of the waveform of a pulse.

At step 1206, the waveform may be determined. As one example, signal processing may take place on the information received from the transducer. As another example, a graph may be plotted, points in time may be examined or any other mathematical algorithm may be applied to the received information such that the waveform and/or aspects of the waveform may be available for processing. Accordingly, step 1206 may comprise means for determining the waveform.

At step 1208, the determined waveform may be compared with a predetermined waveform. In one embodiment, the predetermined waveform may be a waveform included in one or more memories or databases. As another example, the predetermined waveform may be a waveform that was previously detected and stored. Regardless, the predetermined waveform will serve as a bases for comparison to the measured waveform. Accordingly, step 1208 may comprise means for comparing the determined waveform with a predetermined waveform.

At step 1210, misalignment and/or distance to the artery may be determined based on the comparison of the determined waveform with the predetermined waveform. As one example, a computing element may perform one or more statistical basis of comparison and assign error ranges for each of the basis of comparison. Accordingly, the computing element may be able to determine if the comparison is within a range to indicate the alignment of a transducer with the artery. Thus, as one example, the distance from the transducer to the artery may be determined, while in another example, a vector indicating the direction of alignment may be determined. Accordingly, step 1210 comprises means for determining misalignment based on the comparison.

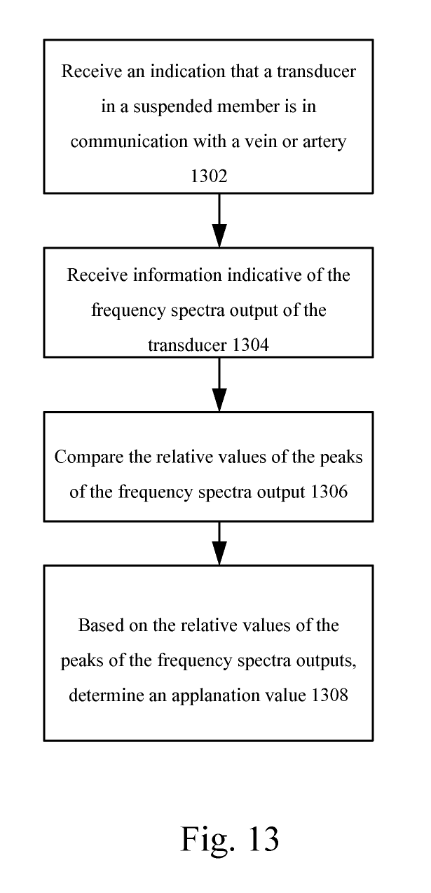

FIG. 13 depicts an example method for optimizing the applanation of a vein or artery. At step 1302, an indication that a transducer in a suspended member is in communication with a vein or artery may be received. As noted above, this may comprise a computing device, and one or more wires or other transmission means. Accordingly, step 1302 comprises means for receiving an indication that a transducer in a suspended member is in communication with a vein or artery.

At step 1304, information indicative of the frequency spectra output of the transducer may be received. The transducer may be similar to transducer 220 described above. Accordingly, step 1304 comprises means for receiving information indicative of the frequency spectra output of the transducer.

At step 1306, the relative values of the peaks of the frequency spectra output 1306 may be compared. As shown in FIGS. 5(a)-(b), frequency output may comprise a plurality of peaks in the frequency. A computing device may comprise logic, coding, instructions and the like configured to determine the peak values of the one or more peaks and compare them. Accordingly, step 1306 comprises means for comparing the relative values of the peaks of the frequency spectra.

At step 1308, applanation of a vein or artery may be determined based on the relative values of the peaks of the frequency spectra outputs. A computing device may comprise logic, coding, instructions and the like configured to determine the amount of applanation based on the relative values. Accordingly, step 1308 comprises means for determining the applanation of a vein or artery based on the relative values of the peaks of the frequency spectra.

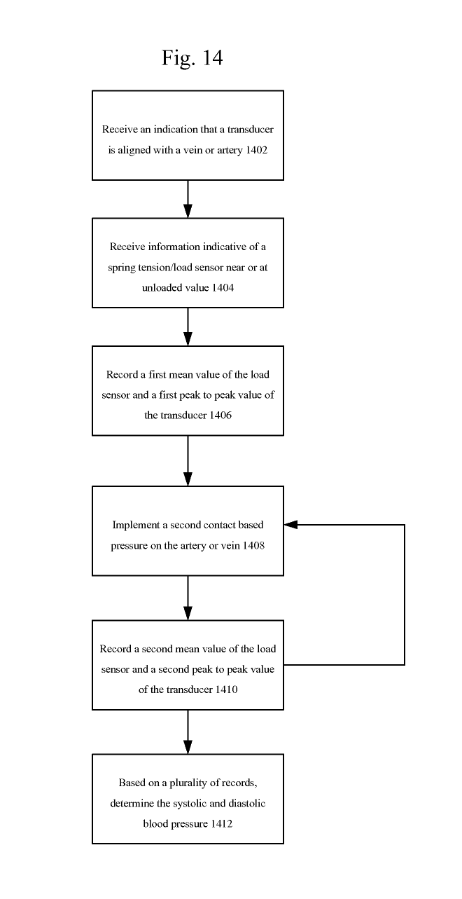

FIG. 14 depicts an example calibration and measurement method. At step 1402, an indication that a transducer is aligned with a vein or artery may be received. This may include a manual indication or data or information received from an earlier alignment procedure. Accordingly, step 1402 comprises means for receiving an indication that a transducer is aligned with a vein or artery.

At step 1404, information indicative of a spring tensor/load sensor output at or near unloaded value. For example, this may be an non applanated state, or an unloaded state of a clamp, actuator, and the like. This may provide a starting point. Accordingly, step 1404 comprises means for receiving information indicative of a spring tension/load sensor output at or near unloaded value.

At step 1406, the peak to peak value of the pulse as measured and output by the transducer and the means value of the load sensor may be determined, recorded and saved. This may comprise a computing element and one or more processes, instructions, logic and the like. Accordingly, step 1406 comprises means for recording the mean value of the load sensor and the peak to peak value as measured and output by the transducer.

At step 1408, the contact based pressure on the artery or vein may be increased. This may comprise actuation of the spring, the suspended member or the like as described above. Accordingly, step 1408 comprises means for increasing the contact based pressure on the artery or vein.

At step 1410, the mean value of the load sensor and the peak to peak value as measured and output by the transducer may again be determined. This may be a looping process in small steps providing a detailed output of the mean value of the load sensor and the peak to peak value as measured and output by the transducer over a range of contact based pressures. Accordingly, in one embodiment, this output may be displayed as a graph, such as the graph depicted in FIG. 7. Further, one or more calculations, processes and the like may take place to determine the diastolic pressure and systolic pressure as indicated on the graph in FIG. 7 and noted at step 1412. Accordingly, step 1410 may comprise means for recording the mean value of the load sensor and the peak to peak value as measured and output by the transducer, and step 14 12 may comprise means for determining the systolic and diastolic blood pressure based on the plurality of records.

It is important to note that once the depiction of FIG. 14 is complete, the system can use the data as part of a calibration process, taking into account the pre stress, the alignment and the applanation. Accordingly, with the systolic and diastolic blood pressure points and other information, accurate real time measurements may be taken.

Additionally, the subject matter of the present disclosure includes combinations and subcombinations of the various processes, methods, means for, systems and configurations, and other features, functions, acts, and/or properties disclosed herein, as well as equivalents thereof.

### Theory

In a non-limiting example, the equations and methods below may be used for calibration. In an embodiment, an artery or vein 210 may undergo a level of applanation, such as that shown in FIG. 4(b). The load placed on the transducer 220 and the load sensor 222 is measured by the load sensor. The transducer 220 may receive one or more signals associated with the pressure in artery or vein 210 and with the load. The output of the transducer 220 and load sensor 222 may be sent to the computing device and the output may be recorded over time. As such, the computer may be able to determine the peak and trough output of the load sensor 222 and the transducer 220. The computing device may further determine the difference between the peak and the trough of the outputs. The time average of the outputs may also be determined. Further still, the offset may be subtracted from the peak, the trough, and the time average values. The contrast between signal output of the trough and the peak amplitudes (P₀₂) and time average of the signal output from the transducer 220 after subtracting the offset (V_{b02}) and the time average of the signal output from the load sensor 222 after subtracting the offset (Vₜ₀₂).

In an embodiment, the values determined above may be used to find the applanated blood pressure (APB). As non-limiting example, the equations below may be used.

$$\begin{matrix}ABP = \frac{A}{D} ( {V}_{b} - {V}_{bo 2} - C \times ( {\overline{V}}_{t} - {V}_{to 2} ) ) + B & Equation 1 \\ ABP = A ( \begin{matrix}\begin{matrix}( {V}_{b} - {\overline{V}}_{b} ) \\ D\end{matrix} & \vert \begin{matrix}{\overline{V}}_{b} & {V}_{bo 2} & C \times ( {\overline{V}}_{t} & {V}_{to 2} )\end{matrix} )\end{matrix} ) \vert B & Equation 2\end{matrix}$$

In the above equations, V_{b} is the voltage corresponding to the troughs or the peaks, V̅_{b} is the time average of the signal from the transducer 220, V̅ₜ is the time average of the signal from the load sensor 222, A, B are multiplication factors and C is a parameter corresponding to the amount of the signal from the transducer 220 to the load sensor 222 as a result of the displacement of the suspended member 202. D is a factor indicative of the change in contrast between the troughs and peaks in the signal output from the transducer 220.

$$D = 1 + \frac{( P - {P}_{o 2} )}{{P}_{o 2}} \qquad Equation 3$$

As on example, in the equations above, P is the difference between the trough and peaks in the signal from the transducer 220.

In an embodiment, A, B, and C can be determined. C corresponds to the amount of signal transferred from the transducer 220 to the load sensor 222 as a result of the displacement of the suspended member 202. Accordingly, C is dependent on the spring constant of the resilient member having a spring constant 204. Further, C may include the change in the amplitude of the signal at the transducer 220 as well as the change in the amplitude of the corresponding change in the signal from the load sensor 222.

C=ΔVₜ/ΔV_{b} Equation 4:

Further to the above, C may also depend on the anatomical structure at the contact surface 208. For example, softer contact surfaces 208 may have a different C value than harder contact surfaces 208. Accordingly, the determination of C may be made on the fly upon contact with known spring constants or other values.

In an embodiment, A and B above may be used to correlate signals output from the load sensor 222 and the transducer 220. Reference systolic and diastolic blood pressure values may be used to determine A and B. During a calibration procedure, equations 1 and 2 above may be used to solve for A and B. As another example, the systolic and diastolic blood pressure values are used in equation 2 along with the signal amplitudes at the peak and the trough respectively obtained from transducer 220 and the time average of the signal obtained from the load sensor 222. Thus, a pair of equations, one for systolic blood pressure and on for diastolic blood pressure may be used to determine A and B.

As noted above, the suspended member at the contact surface may comprise a protrusion and a sensor area. The force on the protrusion may be Fh and the force on the sensing area may be Fs. The surface area of the protrusion can be Sh and the surface area of the sensing area may be Ss. The blood pressure is Bp and the tissue pressure is Tp. Thus:

Fh=Sh*Bp+Sh*Tp and Fs=Ss*Bp+Ss*Tp

These two linear equations have two unknowns, the blood pressure, and the tissue pressure. Solving these equations, we determine that, by measuring the tissue pressure (called the surface contact pressure above) it is possible to isolate the Blood pressure and the force on the sensor area in a single equation.

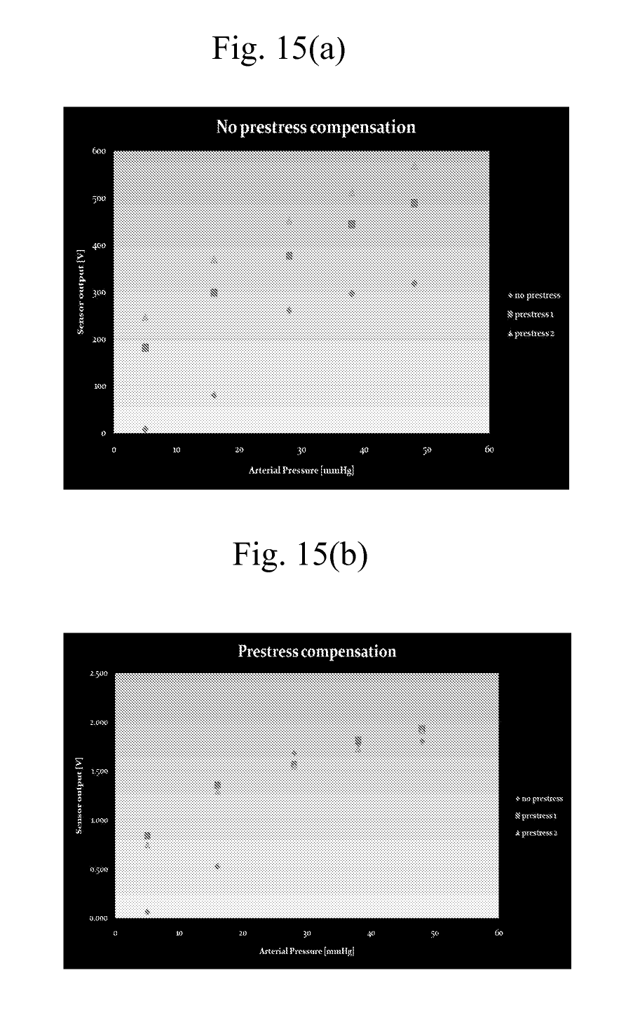

FIGS. 15(a)-(b) depicts a series of experimental results. In FIG. 15(a), results of pressure measurements in a flexible conduit were determined without calibration based on the contact based stress. In FIG. 15(b), the measurements were determined after calibration based on the contact based stress. Also included in both graphs are the actual pressure measurements independently measured. The calibrated measurements had much more accurate results.

1. A blood pressure measurement system comprising:
a reference;
a suspended member coupled to the reference by a resilient member having a spring constant, wherein displacement of the suspended member gives rise to a load on the resilient member having a spring constant;
a load sensor configured to determine the load on the resilient member having a spring constant; and
a processor comprising a computer readable storage medium having stored thereon logic configured to correlate the load on the resilient member having a spring constant to a contact based stress causing the displacement of the suspended member.

2. The system of claim 1 further comprising a sensor area coupled to a transducer and wherein the sensor area and transducer comprise the suspended member.

3. The system of claim 2 wherein the transducer is configured to output a signal correlated with a pulse incident on the sensor area.

4-5. (canceled)

6. The system of claim 1 further comprising an actuator in communication with the resilient member having a spring constant, the actuator configured to place a second load on the resilient member having a spring constant in a direction substantially perpendicular to the surface contact of the sensor area.

7-15. (canceled)

16. The system of claim 1 wherein the computer readable medium comprises logic to determine systolic and diastolic blood pressure based on the peak to peak values of the transducer and mean values of the load sensor at a plurality of surface contact pressures.

17. A method for calibrating a blood pressure sensor comprising:
receiving a first signal from a transducer coupled to a sensor area of a suspended member, which is suspended with respect to a reference by reason of a resilient member having a spring constant;
receiving a second signal from a load sensor configured to measure the load on the resilient member having a spring constant;
determining a first stress from the first signal;
determining a second stress from the second signal; and
adding the second stress to the first stress.

18. The method of claim 17 wherein the load on the resilient member having a spring constant is correlated with a displacement of the suspended member.

19-20. (canceled)

21. The method of claim 17 wherein the suspended member comprises the sensor area.

22. (canceled)

23. The method of claim 17 wherein the first signal is correlated with a pulse incident on the sensor area.

24. (canceled)

25. The method of claim 17 wherein determining further comprising determining an applanation value comprises by:
determining a first pressure value with said sensor area;
receiving a indication that an actuatort has actuated the suspended member;
determining a second pressure value with said sensor area; and
comparing the first pressure value with the second pressure value.

26. (canceled)

27. A blood pressure measurement system comprising:
a reference;
a suspended member coupled to the reference by a resilient member having a spring constant, wherein displacement of the suspended member gives rise to a load on the resilient member having a spring constant; and
a load sensor configured to determine the load on the resilient member having a spring constant.

28. The system of claim 27 further comprising a sensor area coupled to a transducer and wherein the sensor area and transducer comprise the suspended member.

29. The system of claim 28 wherein the transducer is configured to output a signal correlated with a pulse incident on the sensor area.

30-31. (canceled)

32. The system of claim 27 further comprising an actuator in communication with the resilient member having a spring constant, the actuator configured to place a second load on the resilient member having a spring constant in a direction substantially perpendicular to the surface contact of the sensor area.

33-34. (canceled)

35. The system of claim 34 wherein the computer readable storage medium has stored thereon logic configured to determine the applanation of a fluid conduit, and wherein the logic configured to determine the applanation of the fluid conduit comprises logic to measure a first output of the transducer; actuate the resilient member having a spring constant; measure a second output of the transducer; and compare the first output with the second output.

36. The system of claim 35 wherein the computer readable storage medium has stored thereon logic configured to actuate the resilient member having a spring constant based on the applanation of the fluid conduit.

37. The system of claim 33, wherein the computer readable storage medium has stored thereon logic configured to determine the applanation of a fluid conduit, and wherein the logic configured to determine the applanation of the fluid conduit comprises logic to receive the frequency spectra output, determine peaks in the frequency spectra output and compare the relative values of at least two peaks of the frequency spectra output.

38-39. (canceled)

40. The system of claim 39, wherein the signal correlated with the pulse incident on the sensor area provides an indication of alignment of the transducer to the fluid conduit and wherein a first waveform of the pulse incident on the sensor area is compared to a predetermined second waveform to determine the alignment of the transducer to the fluid conduit.

41. The system of claim 27 wherein the computer readable medium has logic to determine peak to peak values of the transducer and mean values of the load sensor at a plurality of surface contact pressures.

42. The system of claim 41 wherein the computer readable medium comprises logic to determine systolic and diastolic blood pressure based on the peak to peak values of the transducer and mean values of the load sensor at the plurality of surface contact pressures.

43-49. (canceled)
